(19) United States
(12) Patent Application Publication
(10) Pub. No.: US 2023/0264044 A1
(43) Pub. Date: Aug. 24, 2023

(54) Convection-Enhanced Diffusing Alpha-Emitter Radiation Therapy

(71) Applicant: ALPHA TAU MEDICAL LTD., Jerusalem (IL)

(72) Inventors: Lior Arazi, Tel Aviv (IL); Itzhak Kelson, Tel Aviv (IL); Ronen Segal, Jerusalem (IL); Raphael Aryeh Levy, Givat Shmuel (IL)

(21) Appl. No.: 18/113,097

(22) Filed: Feb. 23, 2023

Related U.S. Application Data
(60) Provisional application No. 63/313,280, filed on Feb. 24, 2022.

(51) Int. Cl. A61N 5/10

(52) U.S. Cl. CPC A61N 5/1027; A61N 2005/1021

(57) ABSTRACT

A device for treating a tumor, including a needle for insertion into the tumor, a solution generator configured to generate a liquid solution including free radionuclides at a concentration of at least 1*10⁶ atoms per microliter, and a pump which continuously pumps the generated solution into the needle.

### CROSS-REFERENCE TO RELATED APPLICATIONS

The present application claims the benefit of U.S. Provisional Application 63/313,280, entitled “Convection-Enhanced DaRT (CE-DaRT)”, filed on Feb. 24, 2022, whose disclosure is incorporated herein by reference in its entirety.

### FIELD OF THE INVENTION

The present invention relates generally to radiotherapy and particularly to apparatus and methods for providing delivery of radiation therapy to tumors.

### BACKGROUND

Many treatments have been suggested for treating cancer. Some treatments involve delivery of drugs or other agents to a tumor.

U.S. Pat. No. 5,720,720, to Laske et al., the disclosure of which is incorporated herein by reference, describe a convection-enhanced method of drug delivery.

Ionizing radiation is commonly used in the treatment of certain types of tumors, including malignant cancerous tumors, to destroy their cells. Alpha particles are a powerful means for radiotherapy since they induce clustered double-strand breaks on the DNA, which cells cannot repair. Unlike conventional types of radiation, the destructive effect of alpha particles is also largely unaffected by low cellular oxygen levels, making them equally effective against hypoxic cells, whose presence in tumors is a leading cause of failure in conventional radiotherapy based on photons or electrons. In addition, the short range of alpha particles in tissue (less than 100 micrometers) ensures that if the atoms which emit them are confined to the tumor volume, surrounding healthy tissue will be spared.

Diffusing alpha-emitters radiation therapy (DaRT), described for example in U.S. Pat. No. 8,834,837 to Kelson, extends the therapeutic range of alpha radiation, by using radium-223 or radium-224 atoms, which generate chains of several radioactive decays with a governing half-life of 3.6 days for radium-224 and 11.4 days for radium-223. In DaRT, the radium atoms are attached to a source (also referred to as a “seed”) implanted in the tumor with sufficient strength such that they do not leave the source in a manner that they go to waste (by being cleared away from the tumor through the blood), but a substantial percentage of their daughter radionuclides (radon-220 in the case of radium-224 and radium-219 in the case of radium-223) leave the source into the tumor, upon radium decay. These radionuclides, and their own radioactive daughter atoms, spread around the source by diffusion up to a radial distance of a few millimeters before they decay by alpha emission. Thus, the range of destruction in the tumor is increased relative to radionuclides which remain with their daughters on the source.

Given the measured diffusion lengths across a wide range of tumor types, seeds are placed in a tight geometrical arrangement, with typical inter-seed spacing of 3.5-5 mm, to achieve increased effectivity. Depending on the tumor size, a given treatment may involve dozens, or even hundreds, of seeds, with multiple penetration points.

US patent publication 2022/0273832 to Stein et al., titled “Radiolabeled Liposomes and Methods of Use Thereof”, describes convection-enhanced delivery of radiolabeled liposomes.

US patent publication 2011/0135569, titled: “Method for Therapeutic Administration of Radionucleosides”, describes direct infusion under pressure of auger-electron-emitting radio-nucleosides.

### SUMMARY

There is therefore provided in accordance with some embodiments of the invention, a method of treating a tumor, comprising identifying a tumor, and injecting a liquid including free radionuclides into the tumor, for a duration of at least 6 hours.

Optionally, identifying the tumor comprises identifying a tumor having a largest diameter of at least 2 centimeters and wherein injecting the liquid comprises injecting through a single needle. Optionally, injecting the liquid comprises injecting for a duration of at least 72 hours or even at least 110 hours. In some embodiments, injecting the liquid comprises injecting a liquid including beta-emitting radionuclides. Alternatively or additionally, injecting the liquid comprises injecting a liquid including alpha-emission-treatment radionuclides. Optionally, injecting the liquid comprises injecting a liquid including ²¹²Pb radionuclides. Optionally, injecting the liquid comprises injecting a liquid including at least 2*10⁷ radionuclides of an alpha-emission-treatment isotope per microliter. Optionally, injecting the liquid comprises injecting a liquid which does not include more than negligible amounts of ²²⁴Ra, ²²⁰Rn, and ²¹⁶Po. Optionally, injecting the liquid comprises injecting at a rate of at least 2.5 microliters per minute and/or at a rate of not more than 10 microliters per minute.

Optionally, injecting the liquid comprises pumping a liquid through a vial holding therein one or more seeds with parent radionuclides attached to the seeds in a manner that the parent radionuclides do not leave the seed, but a substantial percentage of daughter nuclides leave the seeds into the liquid, upon decay. Optionally, the one or more seeds carry at least 10 microcurie of ²²⁴Ra radionuclides, at least 100 microcurie of ²²⁴Ra radionuclides, or even at least 250 microcurie of ²²⁴Ra radionuclides.

Optionally, the method further includes acquiring an image of the dispersion of the radionuclides in the tumor, determining whether an area of the tumor is not receiving a sufficient number of radionuclides responsive to the acquired image, and adjusting a parameter of the injecting responsive to determining that an area of the tumor is not receiving a sufficient number of radionuclides. Optionally, at least some of the radionuclides are injected coupled to nanoparticles which serve as contrast agents which appear in the acquired image. Optionally, adjusting the parameter comprises changing a flow rate of injecting the liquid. Optionally, adjusting the parameter comprises changing a concentration of the radionuclides in the liquid.

There is further provided in accordance with some embodiments of the invention, a device for treating a tumor, comprising a needle for insertion into the tumor, a solution generator configured to generate a liquid solution including free alpha-emission-treatment radionuclides at a concentration of at least 1*10⁶ atoms per microliter, and a pump which continuously pumps the generated solution into the needle.

Optionally, the device further comprises a controller which is programmed to control the pump to pump the liquid solution through the vial into the needle continuously for at least 24 hours. Optionally, the controller is programmed to control the pump to pump the liquid solution through the vial at a rate of at least 2.5 microliters per minute. Optionally, the controller is programmed to control the pump to pump the liquid solution through the vial at a rate of at least 10 microliters per minute. Optionally, the solution generator is configured to generate the liquid solution with ²¹²Pb radionuclides at a concentration of at least 2*10⁹ atoms per microliter. Optionally, the solution generator comprises a vial including therein one or more seeds with radium radionuclides attached to the seeds in a manner that the radium radionuclides do not leave the seed, but a substantial percentage of daughter radionuclides leave the seeds upon decay and a tube connecting the vial to the needle, wherein the pump is configured to pump a liquid through the vial, into the tube, so as to generate the solution. Optionally, the one or more seeds carry at least 10 microcurie of ²²⁴Ra radionuclides, at least 100 microcurie of ²²⁴Ra radionuclides or even at least 250 microcurie of ²²⁴Ra radionuclides. Optionally, the solution generator is configured to generate a liquid solution including ²¹²Pb atoms at a concentration of at least 1*10⁸ atoms per microliter.

There is further provided in accordance with some embodiments of the invention, a solution for treating a tumor, comprising a bio-compatible liquid solution including free ²¹²Pb atoms at a concentration of between 1*10⁶ and 1*10¹¹ atoms per microliter. Optionally, the solution includes ²¹²Pb atoms at a concentration of at least 1*10⁷ atoms per microliter. Optionally, the solution includes ²¹²Pb atoms at a concentration of at least 1*10⁸ atoms per microliter. Optionally, the solution includes ²¹²Pb atoms at a concentration of less than 2*10⁹ atoms per microliter. Optionally, the solution includes ²¹²Pb atoms at a concentration of at least 1*10⁹ atoms per microliter. Optionally, the solution is suitable for convection-enhanced delivery of the ²¹²Pb atoms.

### BRIEF DESCRIPTION OF THE DRAWINGS

FIG. 1 is a schematic illustration of a radiotherapy solution injection system, in accordance with an embodiment of the present invention;

FIG. 2 is a flowchart of a method of convection enhanced radiotherapy, in accordance with an embodiment of the present invention;

FIG. 3 shows the effect of varying the flow from 0 to 10 μL/min, for a constant source activity of 300 μCi ²²⁴Ra and a treatment duration of five days, in accordance with an embodiment of the present invention;

FIG. 4 shows the effect of varying the source activity from 10 to 1000 μCi ²²⁴Ra for a constant flow of 5 μL/min and a treatment duration of five days, in accordance with an embodiment of the present invention;

FIG. 5 shows the effect of varying the source activity from 30 to 3000 μCi ²²⁴Ra for a constant flow of 5 μL/min for a 12-hour treatment, in accordance with an embodiment of the present invention; and

FIG. 6 shows the treatment duration for a source activity of 300 μCi ²²⁴Ra and a constant flow of 5 μL/min, in accordance with an embodiment of the present invention.

### DETAILED DESCRIPTION OF EMBODIMENTS

An aspect of some embodiments of the invention relates to delivery of radionuclides in a solution into a tumor by enhanced convection delivery. The solution is injected continuously over a relatively long period of at least several hours. The alpha-emitting radionuclides in the solution are free in a manner which allows them and/or their daughter radionuclides to flow with the convection in the tumor. The term free refers to the radionuclides as not coupled to large particles (e.g., particles having a diameter greater than 100 nanometers) which interfere with flow between cells, not coupled to targeting elements (e.g., antibodies) which couple to cells, and not coupled to vectors which are internalized into cancer cells (e.g., liposomes, radionucleosides).

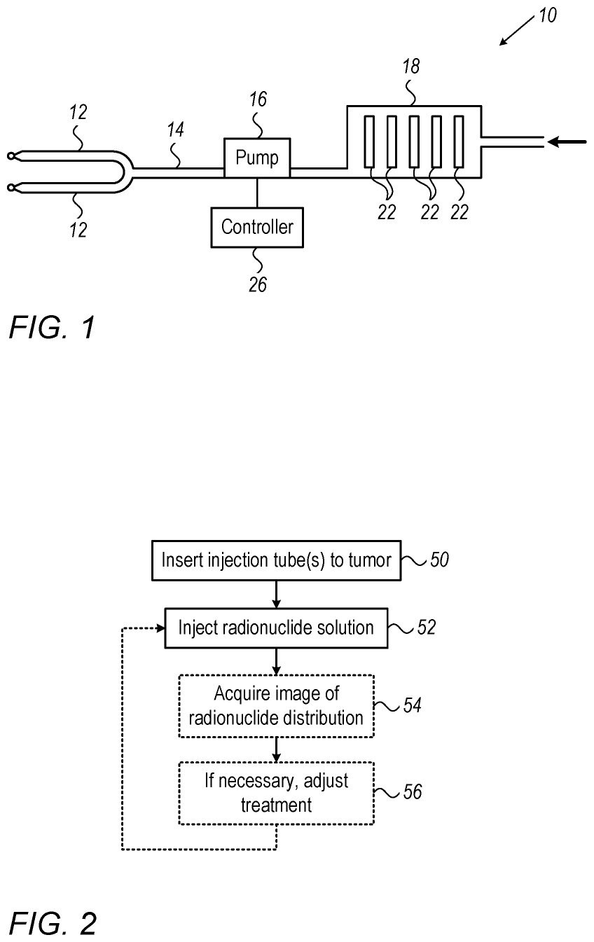

FIG. 1 is a schematic illustration of a radiotherapy solution injection system 10, in accordance with an embodiment of the present invention. System 10 comprises one or more injection tubes 12, delivery tubes 14, a pump 16 and a radionuclide-solution source 18.

Injection tubes 12 are optionally hollow needles designed for implantation in a tumor for a period of at least several hours or even several days. Alternatively or additionally, injection tubes 12 comprise catheters. Delivery tubes 14 are designed to lead a radionuclide-solution from source 18 to injection tubes 12 and into the tumor.

In some embodiments of the invention, the number of injection tubes 12 used depends on the size of the tumor. Optionally, the minimal number of injection tubes 12 which cover the entire tumor is used, in order to minimize the number of points of entrance into a patient having the tumor being treated. The layout of outlets of injection tubes 12 is optionally selected such that every point in the tumor is not farther than a predetermined maximal distance from at least one of the outlets. In some embodiments, the predetermined maximal distance is at least 5 millimeters, at least 7 millimeters, at least 9 millimeters, at least 11 millimeters or even at least 13 millimeters. It is noted that in the use of DaRT seeds the maximal distance used is about 1.5-2.5 millimeters. Thus, the present method reduces significantly the number of penetrations into the patient, relative to other radiotherapy methods. Optionally, though, the maximal distance in some embodiments is smaller than 15 millimeters, smaller than 13 millimeters, smaller than 11 millimeters, or even smaller than 10 millimeters to ensure sufficient radiation throughout the tumor. Accordingly, even large tumors can be covered by less than 10 injection tubes 12, less than 6 injection tubes 12 or even by less than 4 injection tubes 12. In many cases only a single injection tube 12 is used. In other embodiments, at least 2 or at least three tubes or used as a minimum to provide robustness in case one of injection tubes 12 gets clogged or out of place. In some embodiments, the same flow rate of the solution and the same radionuclide concentration are provided through all of the one or more injection tubes 12. Alternatively, the flow rate and/or radionuclide concentration of one or more of the injection tubes 12 is adjustable separately from that of the other injection tubes 12.

FIG. 2 is a flowchart of a method of treating a patient using system 10, in accordance with an embodiment of the present invention. The method includes inserting (50) the one or more injection tubes 12 into the tumor, and operating system 10 to continuously inject (52) the radionuclide-solution into the tumor. During the injection (52), pump 16 causes the solution to flow at the designated rate and the solution flows radially outward from outlets of injection tubes 12 and disperses in the tumor. In some embodiments, after an initial period, one or more images of the treated tumor are acquired (54) in order to verify the distribution of the radionuclides in the tumor. If necessary, the treatment is adjusted (56) based on the acquired images. The injection of the radionuclide solution then continues, with the adjusted treatment, if adjustments were made, or with the original parameters if adjustments were not made. Optionally, the acquiring (54) of images and treatment adjustment (56) is repeated periodically.

In some embodiments, system 10 and/or the method of FIG. 2 are used to treat a malignant tumor in a location which is difficult to access, for example in a deep-seated organ. For example, system 10 and/or the method of FIG. 2 are used to treat a tumor in the brain (e.g., a glioblastoma tumor, brain metastases of other tumor types), lungs, pancreas and/or kidneys. In further embodiments, system 10 and/or the method of FIG. 2 are used to treat large tumors, for example sarcoma. It is noted, however, that system 10 and/or the method of FIG. 2 may be used for any other type of tumor.

System 10 optionally drives the radionuclide-solution into the tumor at a constant rate or a close to contrast rate, for example within 20%, within 10% or within 5% of a base rate. The constant or base rate is optionally at least 0.1 microliters per minute, at least 0.5 microliters per minute, at least 1 microliter per minute, at least 2.5 microliters per minute, or even at least 4 microliters per minute. Generally, the constant or base rate is lower than 50 microliters per minute, lower than 20 microliters per minute, not more than 10 microliters per minute, or even lower than 8 microliters per minute. In one exemplary embodiment, the constant or base rate is of the order of 5 microliters (μL) per minute. Alternatively to a constant rate or a close to constant rate, the radionuclide-solution may be provided in a varying predetermined profile over time, such as a profile with an increasing rate, a decreasing rate, a sinusoidal variation or any other modulation. In some embodiments, the varying predetermined profile over time is designed to compensate for variations in the concentration of radionuclides in the solution, by increasing the flow rate when the activity concentration decreases and/or decreasing the flow rate when the concentration increases.

Pump 16 is optionally controlled by a controller 26 to drive the radionuclide-solution in accordance with the predetermined profile.

It is noted that instead of continuous injection of the radionuclide-solution, the solution may be injected intermittently, for example allowing for a pause every several hours.

In some embodiments, the injected solution includes one or more radionuclides which are alpha-emitting radionuclides or are a parent of a decay chain including at least one alpha-emitter. The term “alpha-emission-treatment radionuclide” is used herein to refer to both alpha-emitting radionuclides and to radionuclides whose decay chain includes an alpha-emission decay, even if the first decay in the chain is not an alpha-emission decay. In other embodiments, the injected solution includes one or more radionuclides which only emit beta radiation.

In some embodiments, the one or more radionuclides are ones which are quickly taken away by the blood circulation in high percentages. This provides a built-in protection against damage to normal tissue around the tumor, as when the radionuclides reach the tumor periphery, they are quickly trapped in red blood cells and taken away by the blood circulation. The one or more radionuclides are optionally trapped in red blood cells, such as lead atoms, (e.g., ²¹³Pb, ²¹²Pb, ²¹¹Pb, ²⁰⁹Pb) atoms, and/or are trapped in blood vessels for other reasons.

Alternatively or additionally, the one or more radionuclides in the injected solution have a decay chain with a longest half-life to dominant emission (i.e., the emission which provides the main treatment) which is not too short, such that it does not lose too much of its activity before reaching the tumor and is not too long so that the percentage of radionuclides that leave the tumor before their dominant emission is not too high. The longest half-life to dominant emission of a radionuclide, is the longest half-life of the radionuclide and of its daughter radionuclides down a decay chain until a last desired emission is reached. For example, the radionuclide of ²¹³Pb has the following decay chain:
²¹³Pb (10.2 min)->²¹³Bi (45.59 min)->²¹³Po (3 microseconds) or ²⁰⁹Ti (2 min)->²⁰⁹Pb (3.253 hours)->²⁰⁹Bi (basically stable)
   The longest half-life to dominant emission in this chain is 45.59 minutes.

Applicant has determined that the average period for which radionuclides remain in a tumor is on the order of several hours. Accordingly, applicant has determined that it is preferred to use an isotope which has a longest half-life to emission of less than 48 hours, less than 24 hours, less than 16 hours, less than 12 hours or even less than 10 hours. On the other hand, in order to maximize the effective range of the treatment from the outlet of injection tube 12, the longest half-life to emission is at least 10 minutes, at least 30 minutes, at least 60 minutes, at least 3 hours, or even at least 5 hours.

In some embodiments, the one or more radionuclides in the injected solution include ²¹²Pb atoms. Alternatively or additionally, the one or more radionuclides in the injected solution comprise ²¹¹Pb atoms. Further alternatively, the one or more radionuclides in the injected solution comprise ²¹³Pb or ²¹³Bi. In still other embodiments, the one or more radionuclides in the injected solution comprise ²²⁴Ra, ²²³Ra and/or ²²⁵Ac.

The base of the injected solution optionally includes distilled water, saline or any other suitable liquid, which is biocompatible and suitable for injection into a tumor, such as liquids known to be used in Convection-Enhanced delivery of drugs.

The radionuclides in the solution are optionally free to flow in the tumor with the convection flow. In some embodiments, when injected, the radionuclides are free from coupling to any other particles. It is noted, however, that in the tumor, the radionuclides may couple to proteins which generally do not interfere with their distribution throughout the tumor. Alternatively, some or all of the radionuclides are coupled to small nanoparticles, which do not target cells, and have a diameter smaller than 40 nanometers, smaller than 30 nanometers, smaller than 20 nanometers, smaller than 10 nanometers, and even smaller than 5 nanometers. This alternative may be used to delay removal of the radionuclides from the tumor and thus increase the percentage of the radionuclides that decay in the tumor. This alternative is particularly desirable for isotopes with a longest half-life of their decay chain, that is greater than 12 hours or even greater than 24 hours. Alternatively or additionally, the nanoparticles or a portion of them (e.g., at least 10%, at least 20%, at least 50%, at least 80%) comprise a material which is easily identified in medical images, such as gold.

Referring in detail to acquiring (54) the image, in some embodiments the image is acquired by gamma imaging. Alternatively, any other suitable modality, such as computed tomography (CT), is used to acquire (54) the image. Optionally, as discussed above, nanoparticles comprising a contrast agent, such as gold, are attached to the radionuclides in order to help in identification of the layout of the radionuclides in the image.

The treatment adjustment (56) may include, for example, upon determining a low level of radionuclides in large areas of the tumor, increasing the flow rate and/or increasing the radionuclide concentration in the solution. When a high level of radionuclides in the tumor is identified, the flow rate and/or the radionuclide concentration may be reduced. Furthermore, when one or more specific areas of the tumor are identified as having a low radionuclide content, an additional injection tube 12 may be inserted into the specific area of the tumor, and/or a solid radiotherapy seed may be implanted in the specific area.

### Radionuclide-Solution Source

In some embodiments, radionuclide-solution source 18 is a reservoir which carries a solution including the radionuclides to be injected. The solution in radionuclide-solution source 18 optionally includes radionuclides at the concentration to be injected. Alternatively, in radionuclide-solution source 18 includes radionuclides at a high concentration and the solution taken out of the reservoir is diluted by pump 16 from a base liquid source (not shown). Optionally, in these embodiments, the solution including the radionuclides is generated separately from system 10, using any method known in the art.

In some embodiments, the solution including the radionuclides is generated using any of the wet-preparation methods described in PCT publication WO 2021/070029 “Wet preparation of Radiotherapy Sources”, the disclosure of which is incorporated herein by reference in its entirety. Alternatively or additionally, any of the methods described in Russian patent 2734429, U.S. Pat. No. 6,126,909 to Rotmensch et al., and/or U.S. Pat. No. 5,038,046 to Norman et al, the disclosures of which are incorporated herein by reference, are used to generate the solution.

In other embodiments, radionuclide solution source 18 comprises a vial containing one or more physical elements 22 carrying one or more parent radionuclides of the one or more radionuclides to be included in the injected solution. Liquid residing in the vial or passing through the vial collects daughter radionuclides of these one or more parent radionuclides. The parent radionuclides are optionally attached to the physical elements 22 with a sufficiently strong bond, such that the parent radionuclides do not leave physical elements 22 with the liquid. On the other hand, the parent radionuclides are optionally attached to the physical elements 22 such that a significant percent (e.g., at least 20%, at least 30%, at least 40% or even at least 50%) of daughter radionuclides generated upon nuclear decay leave the physical elements 22 into the liquid. The parent radionuclides are attached to the physical elements 22 using any suitable method known in the art, such as any of the methods described in U.S. Pat. No. 8,834,837 to Kelson, PCT publication WO 2007/0130260, titled “A radioactive Surface Source and a Method for Producing the Same”, and PCT publication WO 2018/207105, titled: “Polymer Coatings for Brachytherapy Devices”, which are all incorporated herein by reference in their entirety.

The physical elements 22 comprise surface sheets, bars, balls, seeds, wires or any other suitable structure. Optionally, physical elements 22 are placed in radionuclide solution source 18 in a manner which allows replacement during the treatment of a patient. In some embodiments, radionuclide solution source 18 contains a plurality of physical elements 22. Periodically, for example every several hours, one of the physical elements is replaced with a fresh element, in order to increase the activity introduced in the solution and avoid too large a decrease in the activity of the radionuclide solution provided to the patient.

In some embodiments, the parent radionuclides on physical elements 22 comprise ²²⁴Ra, ²²³Ra and/or ²²⁵Ac. The parent radionuclides optionally have an activity of at least 10 microcurie (μCi), at least 50 μCi, at least 150 μCi, at least 250 μCi, at least 500 μCi, at least 800 μCi, at least 1500 μCi, at least 2000 μCi or even at least 3000 μCi. The casing of radionuclide solution source 18 is optionally designed to shield the radiation inside radionuclide solution source 18 according to the level of radiation therein.

### Activity

Different tumors require different amounts of radiation, according to the specific type of tumor. As stated in PCT publication WO 2022/259166, titled: “Activity Levels for Diffusing Alpha-Emitter Radiation Therapy”, which is incorporated herein by reference, applicant has determined the following biological effective dose (BED) of various types of cancer tumors in Gray equivalent (GyE). These dose values are for photon-based radiation (x-, or gamma-rays). Alpha radiation is considered more lethal to cells, and therefore the dose of alpha radiation in Gray is multiplied by a correction factor known as relative biological effect (RBE), currently estimated as 5, to convert it to BED in Gray equivalent (GyE). The BED for alpha emitters is the sum of the alpha dose multiplied by the RBE, and the beta dose.

TABLE 1

| | | Required dose |
|---|---|---|
| | | (Biological effective |
| | Effective long-term | dose (BED) |
| | diffusion length in | in Gray |
| Tumor type | millimeters (all sizes) | equivalent) |
| Squamous cell carcinoma | 0.44 | 60 |
| Colorectal | 0.44 | 120 |
| Glioblastoma (GBM) | 0.27 | 100 |
| Melanoma | 0.40 | 150 |
| Prostate | 0.32 | 173 |
| Breast (triple negative) | 0.35 | 60 |
| Pancreatic cancer | 0.29 | 100 |

In planning a treatment session, it is required to take into account the radionuclides that get washed out of the tumor before they decay. The percentage of radionuclides that are cleared out of the tumor before decay is determined by the ratio between the average time that the radionuclides reside in the tumor and the half-life of the radionuclide.

In using convection enhanced delivery of radionuclides to a tumor, the dispersion of the radionuclides throughout the tumor is mainly due to the convection, such that the diffusion length is not expected to have a significant direct effect on the treatment. In order to destroy a tumor, about 10¹⁰ alpha-emission decays for each cm³ of the tumor are required, assuming even distribution of the decays throughout the tumor. In order to compensate for radionuclides that leave the tumor before decay and for non-even distribution of the radionuclides in the tumor, a larger number of radionuclides is delivered to the tumor during treatment.

For an alpha-emitter radionuclide which has a 50% probability of leaving the tumor before decay, a total dose of at least 1*10¹¹ radionuclides, at least 2.5*10¹¹ radionuclides, at least 5*10¹¹ radionuclides, at least 1*10¹² radionuclides, at least 5*10¹² radionuclides or even at least 1*10¹³ radionuclides is used, for each cm³ of the tumor. On the other hand, in order to avoid systemic damage to the patient, the number of radionuclides expected to leave the tumor before decay is optionally less than 1*10¹³, less than 5*10¹², less than 2*10¹², or even less than 1*10¹². Particularly, when the alpha-emitter radionuclide is Pb-212, the number of radionuclides expected to leave the tumor before decay is less than 2*10¹³ or even less than 2*10¹².

For a beta-emitting radionuclide which has a 50% probability of leaving the tumor before decay, a total dose of at least 1*10¹² radionuclides, at least 2*10¹² radionuclides, at least 1*10¹³ radionuclides or even at least 1*10¹⁴ radionuclides is used, for each cm³ of the tumor. On the other hand, in order to avoid systemic damage to the patient, the number of beta emitting radionuclides expected to leave the tumor before decay is optionally less than 1*10¹⁴, less than 5*10¹³, less than 1*10¹³, less than 5*10¹², or even less than 2*10¹².

### Duration and Concentration

The total number of radionuclides may be provided in a shorter duration with a higher concentration or in a longer duration with a lower concentration. A longer duration is preferred for logistic reasons, in order to avoid the handling of high activity radiation sources in system 10 and the waste of parent radionuclides used to generate the radionuclides delivered to the patient. On the other hand, for the convenience of the patient, a shorter duration may be desired. The treatment is optionally provided for several hours in order to allow for buildup of radionuclides in the tumor. The treatment is optionally provided for at least 6 hours, at least 12 hours, at least 24 hours, at least 48 hours, at least 96 hours or even at least 120 hours.

The injected solution generally carries at least 2*10⁵ radionuclides per second, at least 5*10⁵ radionuclides per second, at least 2*10⁶ radionuclides per second, at least 5*10⁶ radionuclides per second, or even at least 2*10⁸ radionuclides per second. On the other hand, in order to avoid use of too high a concentration, the injected solution optionally includes less than 2*10⁹ radionuclides per second, less than 1*10⁹ radionuclides per second, less than 5*10⁸ radionuclides per second, less than 2*10⁸ radionuclides per second, less than 1*10⁸ radionuclides per second and in some embodiments even less than 5*10⁷ radionuclides per second.

The injected solution optionally has a concentration of at least 1*10⁶ radionuclides per microliter, at least 4*10⁶ radionuclides per microliter, at least 1*10⁷ radionuclides per microliter, at least 1*10⁸ radionuclides per microliter, at least 1*10⁹ radionuclides per microliter, or even at least 1*10¹⁰ radionuclides per microliter. In some embodiments, the injected solution has a concentration of less than 1*10¹¹ radionuclides per microliter, less than 1*10¹⁰ radionuclides per microliter, less than 1*10⁹ radionuclides per microliter or even less than 4*10⁸ radionuclides per microliter.

### Local or Systemic Damage

In some embodiments, the injected solution does not include significant amounts of radionuclides which are not quickly trapped in the blood stream, as these radionuclides may reach and damage healthy tissue surrounding the tumor, in high quantities. Alternatively or additionally, the injected solution does not include significant amounts of radionuclides which have long term half-lives of more than 3 days, such as ²²⁴Ra, and particularly not of more than 8 days, such as ²²³Ra, as these radionuclides may cause systemic damage when provided in large quantities.

Optionally, in these embodiments, the solution does not include more than 1*10⁵ atoms, not more than 1*10 atoms, or even not more than 1*103 atoms per second, of radionuclides which have long term half-lives. For example, when the parent radionuclide comprises ²²⁴Ra, the amount of ²²⁴Ra, ²²⁰Rn and/or ²¹⁶Po in the solution is optionally negligible. Alternatively, higher amounts of these radionuclides are allowed to be included in the injected solution, as long as the number of these radionuclides does not reach a safety limit. In embodiments in which ²²⁴Ra is used as the parent radionuclide, ²¹²Pb and its daughter atoms (²¹²Bi, ²¹²Po and ²⁰⁸Tl) enter the tumor, while ²²⁴Ra and its short-lived daughters ²²⁰Rn and ²¹⁶Po, generally remain outside, or only small even only negligible amounts of the daughters ²²⁰Rn and ²¹⁶Po enter the tumor. The radiation dose inside the tumor is dominated by the alpha decays of ²¹²Bi and ²¹²Po, with additional contribution of beta-electrons from 212Pb, ²¹²Bi and ²⁰⁸Tl.

In other embodiments, the radionuclides in the injected solution include radionuclides which begin a chain of several alpha emissions, despite their relatively long half-life of more than 3 days or more than 8 days, such as ²²⁴Ra, ²²³Ra, or ²²⁵Ac. While more than 90% of the radionuclides may leave the tumor before decay, the additional decays in the chain provide extra destruction of tumor cells which allows for use of lower numbers of radionuclides, below a safety limit for systemic damage.

### Analysis

The spatial fall-off of the dose field is governed by the convective flow of the solution, with negligible contribution by diffusion, and the diffusion to healthy tissue outside the tumor is similar to DaRT from a seed implanted in a tumor. Use of convection allows covering a much larger region through a single penetration point, and reduces the total activity needed to cover the tumor volume.

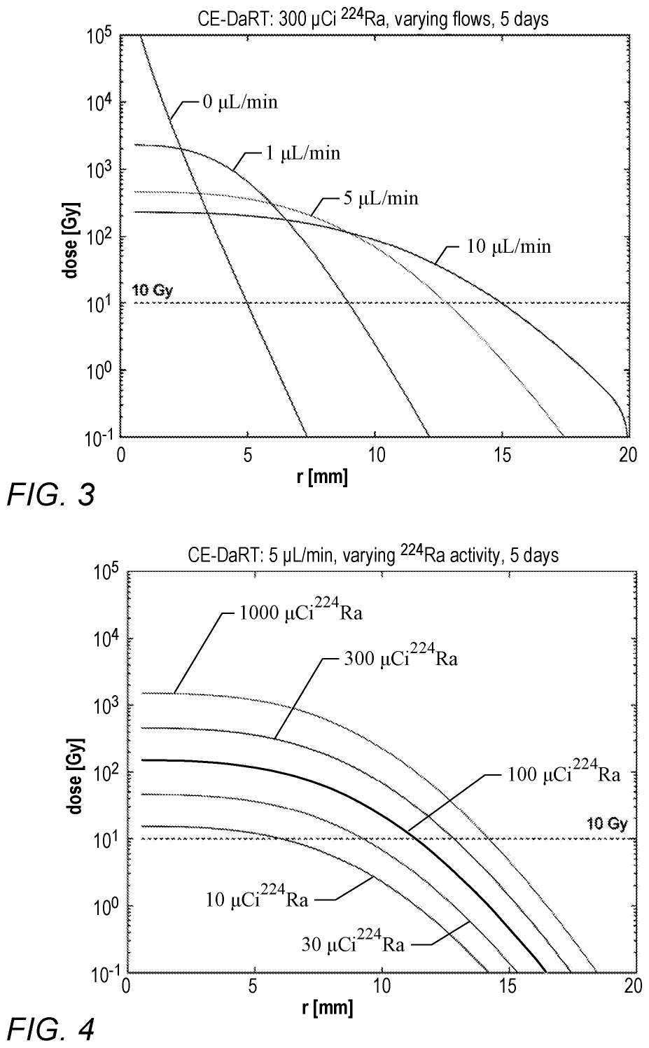

FIGS. 3 and 4 show the results of dose calculations in CE-DaRT, under a simplifying assumption that the system has spherical symmetry (not considering the presence of the needle). The outlet opening of injection tube 12 has a 1 mm diameter. The treatment is assumed to last continuously over 5 days.

FIG. 3 shows the effect of varying the flow from 0 to 10 μL/min, for a constant source activity of 300 μCi ²²⁴Ra in radionuclide-solution source 18, such that the injected solution includes ²¹²Pb.

FIG. 4 shows the effect of varying the source activity from 10 to 1000 μCi ²²⁴Ra for a constant flow of 5 μL/min. The 10 Gy line is shown in both figures, as a reference therapeutic alpha particle dose. As can be seen, for reasonable choices of source activities and flows, one can cover a spherical region with a radius of 10-15 mm from a single entry-point.

FIG. 5 shows the effect of varying the source activity from 30 to 3000 μCi ²²⁴Ra for a constant flow of 5 μL/min for a 12 hour treatment.

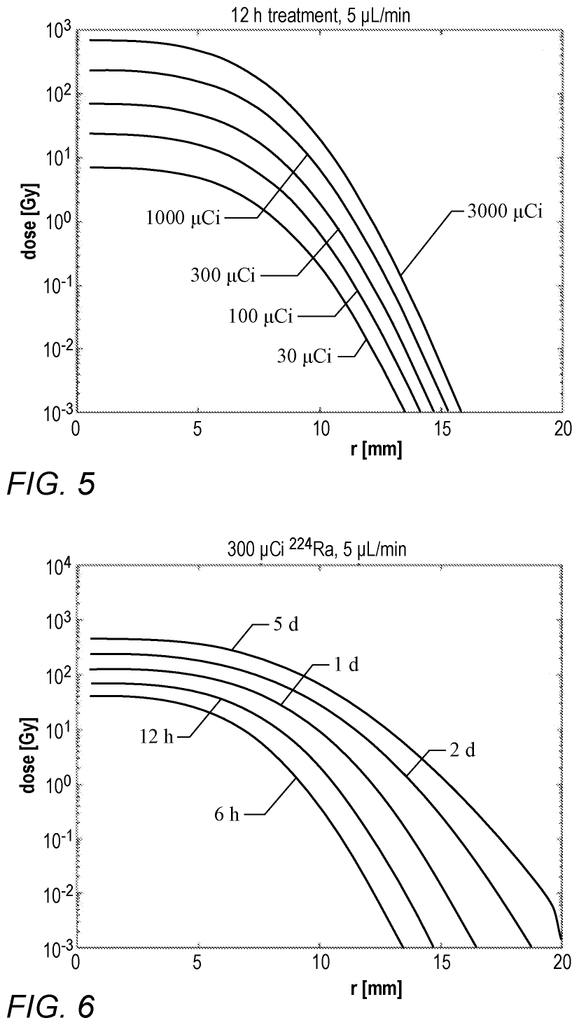

FIG. 6 shows the treatment duration for a source activity of 300 μCi ²²⁴Ra and a constant flow of 5 μL/min.

In some embodiments, in preparation for a treatment session, the size and type of the tumor are determined. According to the size of the tumor, possibly after insertion of injection tube 12, the required radius of treatment is determined. Responsive to the type of the tumor, a required radiation level in Grey is optionally determined. Accordingly, using the graph of FIG. 4 and/or FIG. 5, and based on the required radius of treatment from the outlet of injection tube 12, the radiation level is determined. Alternatively or additionally, using the graph of FIG. 6, the duration of the treatment is selected. Further alternatively or additionally, using the graph of FIG. 3, the flow rate is selected.

### CONCLUSION

It will be appreciated that the above-described methods and apparatus are to be interpreted as including apparatus for carrying out the methods and methods of using the apparatus. It should be understood that features and/or steps described with respect to one embodiment may sometimes be used with other embodiments and that not all embodiments of the invention have all of the features and/or steps shown in a particular figure or described with respect to one of the specific embodiments. Tasks are not necessarily performed in the exact order described.

While the above description relates mainly to free radionuclides not coupled to large particles, not coupled to targeting elements, and not coupled to vectors which are internalized into cancer cells, some embodiments of the invention involve use of radionuclides which are not entirely free. In some of these embodiments, a small percentage (less than 30%, less than 20%, less than 10%) of radionuclides are not free. Alternatively or additionally, the radionuclides are non-free in that they slightly go beyond the definition of free.

It is noted that some of the above-described embodiments may include structure, acts or details of structures and acts that may not be essential to the invention and which are described as examples. Structure and acts described herein are replaceable by equivalents which perform the same function, even if the structure or acts are different, as known in the art. The embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art. Therefore, the scope of the invention is limited only by the elements and limitations as used in the claims, wherein the terms “comprise,” “include,” “have” and their conjugates, shall mean, when used in the claims, “including but not necessarily limited to.”

1. A method of treating a tumor, comprising:
identifying a tumor; and
injecting a liquid including free radionuclides into the tumor, for a duration of at least 6 hours.

2. The method of claim 1, wherein identifying the tumor comprises identifying a tumor having a largest diameter of at least 2 centimeters and wherein injecting the liquid comprises injecting through a single needle.

3. The method of claim 1, wherein injecting the liquid comprises injecting for a duration of at least 72 hours.

4. The method of claim 3, wherein injecting the liquid comprises injecting for a duration of at least 110 hours.

5. The method of claim 1, wherein injecting the liquid comprises injecting a liquid including beta-emitting radionuclides.

6. The method of claim 1, wherein injecting the liquid comprises injecting a liquid including alpha-emission-treatment radionuclides.

7. The method of claim 6, wherein injecting the liquid comprises injecting a liquid including ²¹²Pb radionuclides.

8. The method of claim 1, wherein injecting the liquid comprises injecting a liquid including at least 2*10⁷ radionuclides of an alpha-emission-treatment isotope per microliter.

9. The method of claim 1, wherein injecting the liquid comprises injecting a liquid which does not include more than negligible amounts of ²²⁴Ra, ²²⁰Rn, and ²¹⁶Po.

10. The method of claim 1, wherein injecting the liquid comprises injecting at a rate of at least 2.5 microliters per minute.

11. The method of claim 1, wherein injecting the liquid comprises injecting at a rate of not more than 10 microliters per minute.

12. The method of claim 1, wherein injecting the liquid comprises pumping a liquid through a vial holding therein one or more seeds with parent radionuclides attached to the seeds in a manner that the parent radionuclides do not leave the seed, but a substantial percentage of daughter nuclides leave the seeds into the liquid, upon decay.

13. The method of claim 12, wherein the one or more seeds carry at least 10 microcurie of ²²⁴Ra radionuclides.

14. The method of claim 13, wherein the one or more seeds carry at least 100 microcurie of ²²⁴Ra radionuclides.

15. The method of claim 14, wherein the one or more seeds carry at least 250 microcurie of ²²⁴Ra radionuclides.

16. The method of claim 1, further comprising:
acquiring an image of the dispersion of the radionuclides in the tumor;
determining whether an area of the tumor is not receiving a sufficient number of radionuclides responsive to the acquired image; and
adjusting a parameter of the injecting responsive to determining that an area of the tumor is not receiving a sufficient number of radionuclides.

17. The method of claim 16, wherein at least some of the radionuclides are injected coupled to nanoparticles which serve as contrast agents which appear in the acquired image.

18. The method of claim 16, wherein adjusting the parameter comprises changing a flow rate of injecting the liquid.

19. The method of claim 16, wherein adjusting the parameter comprises changing a concentration of the radionuclides in the liquid.

20. A device for treating a tumor, comprising:
a needle for insertion into the tumor;
a solution generator configured to generate a liquid solution including free alpha-emission-treatment radionuclides at a concentration of at least 1*10⁶ atoms per microliter; and
a pump which continuously pumps the generated solution into the needle.

21. The device of claim 20, further comprising a controller which is programmed to control the pump to pump the liquid solution through the vial into the needle continuously for at least 24 hours.

22. The device of claim 21, wherein the controller is programmed to control the pump to pump the liquid solution through the vial at a rate of at least 2.5 microliters per minute.

23. The device of claim 21, wherein the controller is programmed to control the pump to pump the liquid solution through the vial at a rate of at least 10 microliters per minute.

24. The device of claim 20, wherein the solution generator is configured to generate the liquid solution with ²¹²Pb radionuclides at a concentration of at least 2*10⁹ atoms per microliter.

25. The device of claim 20, wherein the solution generator comprises:
a vial including therein one or more seeds with radium radionuclides attached to the seeds in a manner that the radium radionuclides do not leave the seed, but a substantial percentage of daughter radionuclides leave the seeds upon decay; and
a tube connecting the vial to the needle,
wherein the pump is configured to pump a liquid through the vial, into the tube, so as to generate the solution.

26. The device of claim 25, wherein the one or more seeds carry at least 10 microcurie of ²²⁴Ra radionuclides.

27. The device of claim 26, wherein the one or more seeds carry at least 100 microcurie of ²²⁴Ra radionuclides.

28. The device of claim 27, wherein the one or more seeds carry at least 250 microcurie of ²²⁴Ra radionuclides.

29. The device of claim 20, wherein the solution generator is configured to generate a liquid solution including ²¹²Pb atoms at a concentration of at least 1*10⁸ atoms per microliter.

30. A solution for treating a tumor, comprising:
a bio-compatible liquid solution including free ²¹²Pb atoms at a concentration of between 1*10⁶ and 1*10¹¹ atoms per microliter.

31. The solution of claim 30, wherein the solution includes ²¹²Pb atoms at a concentration of at least 1*10⁷ atoms per microliter.

32. The solution of claim 30, wherein the solution includes ²¹²Pb atoms at a concentration of at least 1*10⁸ atoms per microliter.

33. The solution of claim 32, wherein the solution includes ²¹²Pb atoms at a concentration of less than 2*10⁹ atoms per microliter.

34. The solution of claim 30, wherein the solution includes ²¹²Pb atoms at a concentration of at least 1*10⁹ atoms per microliter.

35. The solution of claim 30, wherein the solution is suitable for convection-enhanced delivery of the ²¹²Pb atoms.
